(19) United States
(12) Patent Application Publication
(10) Pub. No.: US 2007/0093719 A1
(43) Pub. Date: Apr. 26, 2007

(54) Personal heart rhythm recording device

(76) Inventors: Allen Bryant Nichols JR., Falls Church, VA (US); Allen Bryant Nichols SR., Newport News, VA (US)

Correspondence Address: Allen Bryant Nichols Jr., 3003 Cedarwood Ln, Falls Church, VA, 22042, US

(21) Appl. No.: 11/253,984

(22) Filed: Oct. 20, 2005

(51) Int. Cl. A61B 5/04

(52) U.S. Cl. 600/509; 600/523

(57) ABSTRACT

An electrocardiographic device for recording the rhythm of the human heart using a home personal computer and printer. This device consists of three silver-plated leads, a 1000× amplifier, an analog to digital computer, an oscillating timing clock, a microcontroller unit, a USB input bus, a data output bus, and computer software for displaying the rhythm graphically. The advantages of this device include convenience, low cost, and repeatability. A patient can record their cardiac rhythm themselves at any time whenever a sudden cardiac arrhythmia occurs without traveling to the doctor's office or emergency room. Based on the low cost of this inexpensive device a patient can own his own rhythm recording device instead of paying for expensive Holter monitors or event recorders from a doctor's office. Lastly, this device can be used repeatedly without the expense of disposable electrodes or limitations of monitoring device memory restricting the number of electrocardiographic recordings.

### FEDERALLY SPONSORED RESEARCH

Not Applicable

### SEQUENCE LISTING OR PROGRAM

Not Applicable

### BACKGROUND OF THE INVENTION

1. Field of the Invention

This invention relates to electrocardiographic heart rhythm monitoring devices, specifically to inexpensive personal recording of the heart rhythm with a home computer and printer.

2. Prior Art

Several long term rhythm monitoring devices and systems have been developed and marketed over the last thirty years. Previous patents describe different methods for recording EKG signals over periods of time and are primarily designed to provide the physician with methods for assessing patients' disorders over limited periods of time. The monitoring process, as applied to electrocardiography, was named after its inventor and pioneer research physicist, Norman J. Holter, President of the Holter Research Foundation of Helena, Mont. The original Holter concept and invention was assigned to Del Mar Engineering Laboratories of Los Angeles, Calif., under technology license from the Holter Research Foundation and patented with the U.S. Patent Office on Jul. 6, 1962. The application issued as U.S. Pat. No. 3,215,136 on Nov. 2, 1965 and described a long term, ambulatory ECG recording technique and also Holter's data reduction and presentation format. The patent specifically taught a means for processing and recording electrocardiograph signals over a duration of time.

Since 1965, many patents have been granted improving the original Holter patent. On Oct. 31, 1978, U.S. Pat. No. 4,123,785, “Recorder for Cardiac Signals with Manually Activated Event Marker” was granted to Isaac R. Cherry and Donald L. Anderson of Del Mar Avionics, successor to Del Mar Engineering Labs. The patent disclosed a small, hip mounted tape recorder for ambulatory recording of cardiac signals over a twenty-four hour interval and included a clock with visual display and a patient event marker. The cardiac signal is recorded on two tracks simultaneously on magnetic tape. U.S. Pat. No. 4,532,934, was issued August 1985, titled “Pacemaker Monitoring Recorder and Malfunction Analyzer”, by George J. Kelen, M.D. The Kelen patent describes a hip mounted magnetic tape recorder that detects and records sequential pacemaker spikes in one channel in a waveform compatible with corresponding EKG signals recorded in a second tape channel. U.S. Pat. No. 5,109,862 issued May 8, 1992 and was titled, “Method and Apparatus for Spectral Analysis of Electrocardiograph Signals,” by inventors George J. Kelen, M.D. and Raphael Henkin, Ph.D. The Kelen patent disclosed a signal processing and analysis method and apparatus for plotting and measuring ECG signals where the graphic plots and numeric parameters measured reveal abnormalities of electrical conduction within the heart thought to anticipate abnormal heart rhythm, arrhythmia. The invention employs Fourier analysis of short overlapping segments of ECG signal to create a three dimensional electrocardiogram map.

U.S. Pat. No. 5,205,295, issued Apr. 27, 1993 “Method and Apparatus for Holter Recorder with High Resolution Signal Averaging Capability for Late Potential Analysis,” by inventors Bruce Del Mar and Isaac R. Cherry, disclosed a method for digital signal averaging of selected signals and storing for future playback. The average signals, several times per hour in a 24 hour period, are correlated with previously defined correlation coefficients to yield summated results that have eliminated non-repetitive noise.

Del Mar's patent, U.S. Pat. No. 6,117,077, issued Sep. 12, 2000, “Long-term Ambulatory Physiological Recorder,” describes a digital Holter monitor with a USB port. The patent makes further advances with the digital capability of existing technology. The device is small and lightweight, however it still contains memory chips for recording long-term heart rhythms and is priced so that only physicians can purchase the equipment. U.S. Pat. No. 6,701,184, “Virtual Holter,” by Henkin, issued Mar. 2, 2004 describes a method for conducting a worldwide Holter database. Users obtain their Holter recording in a conventional manner, then download the data to a PC. The data is sent through ISP's to a URL web address for a Central Computing Facility for analysis. This patent describes a method to lease rhythm recorders to patients and physicians and charges them to store the data on servers. This method is costly to the patient and does not allow them to view their own heart rhythm, only the post-analysis.

### OBJECTIVES AND ADVANTAGES

Many individuals are concerned about experiencing extra heartbeats, rapid heart or irregular rhythms, skipped heartbeats, or slow heart rates. These cardiac arrhythmias may be associated with palpitations, chest discomfort, light-headedness, shortness of breath, anxiety, or even fainting spells. These symptoms may reflect harmless minor arrhythmias, such as extra-systoles, which cause needless anxiety, or may reflect more serious arrhythmias, such as atrial fibrillation or ventricular tachycardia, which are often indicators of more serious clinically significant heart disease.

Patients with these symptoms have traditionally gone to their primary care physician for advice. The primary care physician, typically an internist or family care practitioner, listens to the patient's description of his or her symptoms, examines the patient, and often records an electrocardiogram (EKG). The EKG may show an abnormal rhythm pattern providing an immediate diagnosis, but usually dysrhythmias are transient and the EKG will be normal. For example, the patient may have experienced several episodes of rapid tachycardia, over several days, but has no tachycardia in the doctor's office. If the EKG is normal, the primary care physician usually then refers the patient to a cardiologist, who has more elaborate equipment for recording abnormal heart rhythms.

The cardiologist usually has in his office two relatively expensive recording devices for detecting arrhythmias: a 24-hour Holter monitoring device and an event recorder. The 24-hour Holter provides a recording of every heart beat during a 24 hour period, and a computer analysis provides a count of each heart beat type and record of heart rates each hour. The event recorder is an electronic monitoring device typically worn by the patient for one week. This device continuously records and erases the patients heart rhythm. If the patient experiences symptoms of a rhythm disorder, he or she presses a button on the recorder that causes a sample of the cardiac rhythm to be stored in the recorder. These recorders are capable of storing only 5 to 30 minutes of rhythm samples, depending on the model of device used. The patient then transmits the events via telephone to the cardiologist's office, where the EKG recordings are printed on paper strips or into a computer, then printed, and finally interpreted by the cardiologist.

The limitations of the Holter monitor and the event recorder are the following: First, patients often experience rhythm disorders only every few weeks or every few months. 24-hour recordings or event recorders for these patients usually fail to detect the arrhythmia. Secondly, the typical sequence of a visit to the primary care physician, followed by multiple visits to the cardiologist is expensive and time consuming for the patient. Thirdly, both Holter monitor recordings and arrhythmia event recordings are expensive, typically costing several hundred dollars for each recording. Fourthly, most patients experience repeated dysrhythmias over months or years and need a practical method for recording cardiac dysrhythmias repeatedly to test the effectiveness of antiarrythmic medications. Fifthly, because the twenty-four hour Holter recording devices and the event recorders both cost several thousand dollars each, the cardiologist cannot loan either device to patients for prolonged periods of time. Sixthly, by directly observing their own heart rhythm on the computer screen, the patient will be able to directly correlate symptoms of palpitations, extra-systoles, tachy or bradyarrhythmia with rhythm abnormalities displayed instantaneously. Finally, patients with implanted cardiac pacemakers can use this recording device for periodically checking their pacemaker function from the comfort of their own home.

We have developed an inexpensive cardiac rhythm recorder device that permits any individual to conveniently record their heart rhythm at home, at anytime using their home computer. This device provides printed electrocardiographic recordings of the heart rhythm that the patient may then take or fax to his or her physician for interpretation of any rhythm disorder and for possible medical treatment with antiarrhythmic agents. The advantages of this device include the following: First, the patient can make their own recordings at anytime day or night without the inconvenience of traveling to the doctor's office during business hours or to the ER nights or weekends. Secondly, with this device, the patient can often obtain a diagnosis of his or her rhythm disorder without the major expense of 24-hour Holter monitors or event recorders. Thirdly, this device will be helpful for patients with chronic recurring arrhythmias over long periods of months to years to help their physicians evaluate the effectiveness of antiarrythmic medications. For example, the physician may instruct his or her patient to record his heart rhythm at home once a week to determine how effective an antiarrythmic drug is for suppressing paroysmal atrial fibrillation.

Fourthly, the inexpensive cost of this rhythm recording device (priced around one-hundred dollars) allows the patient to purchase and own his own recording device, which he can use to record his rhythm himself using his personal computer anytime he experiences tachycardia, palpitations, dizziness, lightheartedness, near-fainting, or other symptoms of dysrhythmia.

This invention relates to an apparatus and digital computer processing for personal, rapid, and inexpensive electrocardiographic recording. More specifically, the invention disclosed herein consists of a handheld sensor for monitoring cardiac electrical signals. This device is designed for convenient and repeated electrocardiograph recordings of cardiac rhythms at home. Only a trained physician can interpret recordings. This device will facilitate convenient, inexpensive recordings of arrhythmias for individual patients and permit the individual to take or fax the electrocardiographic rhythm strips recorded with this device to his physician for interpretation and for medical advice regarding therapy. This device is designed to provide instant recordings of the heart rhythm for subsequent interpretation by a physician; it is not intended for self-diagnosis of cardiac rhythm disorders at home.

The device has three mounted electrode plates in conductance with the skin to sense the electric signals produced by the heart. The signals are then amplified and frequency filtered, and sent via a USB frequency shielded wire to a personal computer. The device contains small, lightweight microchips, and other low-cost widely available electrical circuit components. Since the continual drop in price of electrical circuit components, including the microcontroller itself, the EKG detecting device can be produced very inexpensively. The invention describes how an inexpensive, personal EKG monitoring device for home use with a personal computer and printer can be built.

Using the above electronic components, we have constructed a working model of this cardiac recording device which provides clear electrocardiographic displays of the cardiac rhythm on a personal computer monitor. The rhythms displayed are identical to those recorded in lead two on a conventional twelve-lead EKG.

### SUMMARY

The primary objective of this electrocardiographic recording device is to provide patients with an inexpensive machine for recording their own heart rhythms with their home personal computer and printer. Patients can then take or fax rhythms recorded on paper to their physicians for interpretation and advice regarding medical treatment.

### DRAWINGS—FIGURES

FIG. 1 illustrates the embodiments of the EKG Recorder device used on a human subject.

FIG. 2 illustrates the EKG Recorder device's internal block diagram.

FIG. 3 illustrates a general block flow diagram of the patient's interaction with the device and program.

### DETAILED DESCRIPTION

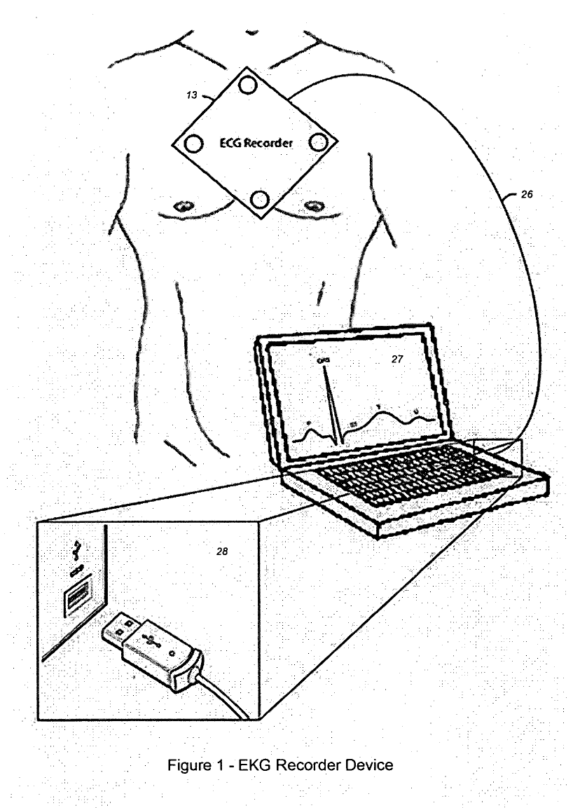

A schematic of the basic setup is shown in FIG. 1. Referring to FIG. 1, the EKG Recorder device 13 is placed on the patient's chest. The EKG Recorder detects the cardiac electromagnetic waves and sends them via a USB cable 26 to a personal computer 27. The personal computer 27 must have a USB input 28 in order for the device to transmit data. Once the data has been received by the personal computer 27, it is analyzed and displayed as a graph via a user-friendly software program.

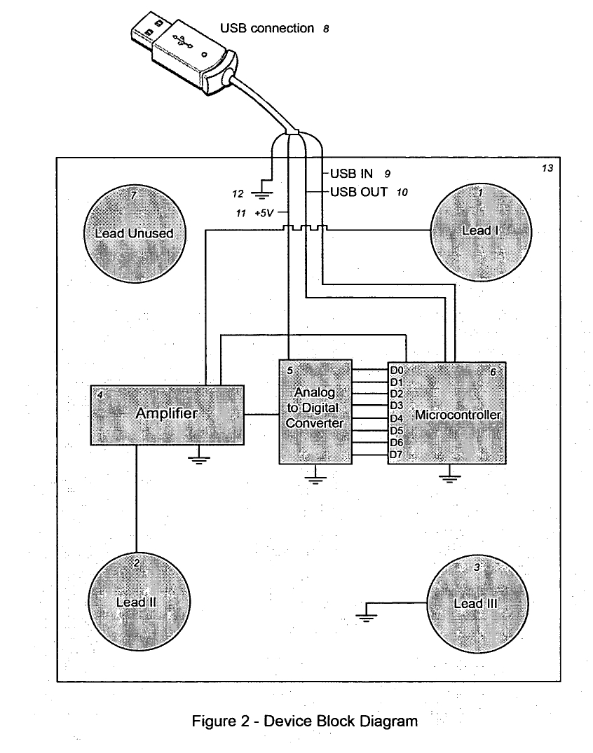

The internal structure of the EKG Recording device is shown in FIG. 2. Referring to FIG. 2, there are three leads 1-3 that detect the electromagnetic signal propagating on the chest. Lead I 1 is used as the positive terminal and Lead II 2 is used as the negative terminal. Lead III 3 is used as the grounding reference. Each lead is made up of identical silver plated material. There is a fourth lead 7 that is used as ground. Lead I 1, Lead II 2, and Lead III 3 each connect to the 1000 gain amplifier 4. The output of amplifier 4 is connected to the input of the analog-to-digital converter 5. The eight line data bus output from the analog-to-digital converter 5 connects to the data input bus of the microcontroller unit 6. A fourth Lead 7 is nonfunctional and is used to help patients stabilize the device.

The USB attachment 8 provides the +5V input 11 for the amplifier 4, the analog-to-digital converter 5, and the microcontroller unit 6. The grounding connection for the components is connected to both Lead III 3 and the USB ground connection 12. The USB attachment 8 provides a data input bus 9 and a data output bus 10 for the microcontroller unit 6. The microcontroller unit 6 is able to communicate with the personal computer FIG. 1-4 with the USB input bus 9 and data output bus 10.

Various other electronics such as; high pass filters, diodes, transistors, capacitors, inductors, potentiometers, resistors, etc. will be implemented into the printed circuit board design. However these electronics are conventional in the art and will not therefore be discussed in detail herein.

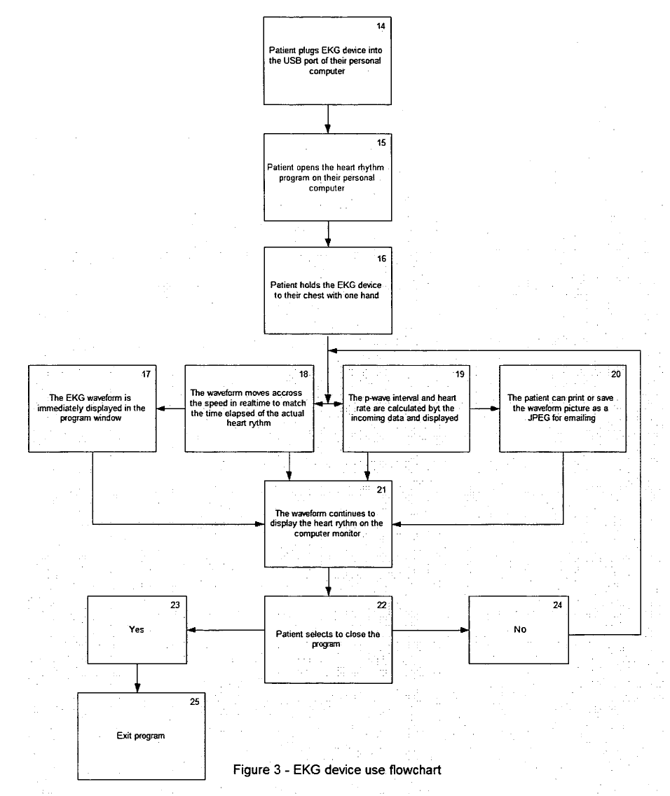

The EKG device usage flowchart is shown in FIG. 3. The patient first plugs the device into their personal computer 14. Then the patient opens up the EKG program on their computer 15. Then the patient holds the device to their chest 16. Inside the program window, the QRS wave is displayed 17, the waveform moves across the screen in real time 18, the p-wave interval and heart rate are displayed 19, and options to print or save the image are available to the user 20. The program continues to display the waveform as long as the device is still held against their chest 21. The patient can select to close the program at any time 22. If the patient chooses to close the program 23, the program exits back to the operating system 25. If the patient wants to continue using the program 24, the program will continue to display the heart rhythm in real time.

We claim:

1. A heart rhythm monitor comprising an electronic device and computer program.

2. The monitor of claim 1 wherein said device contains a reusable array of recording lead electrodes applied to the anterior chest.

3. The monitor of claim 1 wherein said device contains an amplifier for amplifying the electrical voltage recorded from the heart.

4. The monitor of claim 1 wherein said device contains an analog to digital converter.

5. The monitor of claim 1 wherein said device contains an oscillating timing clock.

6. The monitor of claim 1 wherein said device contains a USB data busing microcontroller unit.

7. The monitor of claim 1 wherein said device contains a USB output port to connect to a personal computer.

8. The monitor of claim 1 wherein said computer program displays the cardiac electrical signal in graphical form.

9. The monitor of claim 1 wherein said computer program provides a print command to produce paper records of the heart rhythm.

10. A method of monitoring heart rhythms using a personal computer and printer, comprising:
(a) providing a device of the type comprising of electrical circuit components,
(b) providing said personal computer,
(c) providing said printer,
(d) connecting a USB cable between said device and said personal computer,
(e) opening a computer program to view said heart rhythms,
(f) holding said device to the patient's chest,
(g) viewing and analyzing said heart rhythm inside said computer program,
(h) printing said heart rhythm on said printer,
whereby said device is simple to use for said patients and built with inexpensive, widely available, said circuit components.
